(19) United States
(12) Patent Application Publication
(10) Pub. No.: US 2005/0207531 A1
(43) Pub. Date: Sep. 22, 2005

(54) Radiation therapy system using interior-point methods and convex models for intensity modulated fluence map optimization

(75) Inventors: James F. Dempsey, Gainesville, FL (US); Ravindra K. Ahuja, Gainesville, FL (US); Arvind Kumar, Gainesville, FL (US); Jonathan G. Li, Gainesville, FL (US); H. Edwin Romeijn, Gainesville, FL (US)

Correspondence Address: AKERMAN SENTERFITT, P.O. BOX 3188, WEST PALM BEACH, FL, 33402-3188, US

(73) Assignee: UNIVERSITY OF FLORIDA RESEARCH FOUNDATION, INC., GAINESVILLE, FL (US)

(21) Appl. No.: 11/039,331

(22) Filed: Jan. 20, 2005

Related U.S. Application Data
(60) Provisional application No. 60/537,779, filed on Jan. 20, 2004.

(51) Int. Cl. A61N 5/10

(52) U.S. Cl. 378/65

(57) ABSTRACT

A method of determining a treatment plan for intensity modulated radiation treatment (IMRT) divides a three-dimensional volume of a patient into a grid of dose voxels. At least a portion of the dose voxels are designated to belong to at least one target or to at least one critical structure. An ionizing radiation dose as delivered by a plurality of beamlets each having a beamlet intensity is modeled. A non-linear convex voxel-based penalty function model is provided for optimizing a fluence map. The fluence map defines the beamlet intensities for each of the plurality of beamlets. The model is then solved based on defined clinical criteria for the target and the critical structure using an interior point algorithm with dense column handling to obtain a globally optimal fluence map.

### CROSS-REFERENCE TO RELATED APPLICATIONS

This application claims the benefit of Provisional Patent Application No. 60/537,779 entitled “RADIATION THERAPY SYSTEM USING INTERIOR-POINT METHODS AND CONVEX MODELS FOR INTENSITY MODULATED FLUENCE MAP OPTIMIZATION” filed on Jan. 20, 2004, and is incorporated by reference herein its entirety.

### STATEMENT REGARDING FEDERALLY SPONSORED RESEARCH OR DEVELOPMENT

Not applicable.

### FIELD OF THE INVENTION

The invention relates to intensity modulated radiation therapy (IMRT), and more specifically to a system and method based on optimal planning using convex programming models and interior-point algorithms.

### BACKGROUND

Intensity modulated radiation therapy (IMRT) is a revolutionary type of external beam treatment that is able to conform radiation to the size, shape and location of a tumor. MRT is a major improvement as compared to conventional radiation treatment. The effectiveness of conventional radiation therapy is limited by imperfect targeting of tumors and insufficient radiation dosing. Because of these limitations, conventional radiation can expose healthy tissue to radiation, thus causing complications. With IMRT, the optimal dose of radiation is delivered to the tumor and dose to surrounding healthy tissue is minimized.

In a typical IMRT treatment procedure, the patient undergoes treatment planning x-ray computed tomography (CT) with the possible addition of magnetic resonance imaging (MRI) or a position emission tomography (PET) study. When scanning takes place, the patient is immobilized in a manner consistent with treatment so that the imaging is completed with the highest degree of accuracy. A radiation oncologist or other health care professional typically analyzes these images and determines the areas that need to be treated and areas that need to be spared, such as critical structures including the spinal cord and surrounding organs. Based on this analysis, a safe and effective IMRT treatment plan is developed using large-scale optimization.

IMRT relies on two advanced technologies. The first is inverse treatment planning. Through sophisticated algorithms using high speed computers an acceptable treatment plan is determined using an optimization process which is intended to maximize the dose to the tumor while minimizing exposure to surrounding healthy tissue. During inverse planning a large number (e.g. several thousand) of pencil beams or beamlets which comprise the radiation beam are independently targeted to the tumor or other target structure with high accuracy. Through optimization algorithms the non-uniform intensity distributions of the individual beamlets are determined to attain certain specific clinical objectives.

The second technology comprising IMRT generally utilizes multileaf collimators (MLC). This technology delivers the treatment plan derived from the inverse treatment planning system. A separate optimization called leaf sequencing is used to convert the set of beamlet fluences to an equivalent set of aperture fluences. The MLC is composed of computer-controlled tungsten leaves that shift to form specific patterns, blocking the radiation beams according to the intensity profile from the treatment plan. As an alternative to MLC delivery, an attenuating filter can also be designed to match the fluence of beamlets. After the plan is generated and quality control checking has been completed, the patient is immobilized and positioned on the treatment couch. Radiation is then delivered to the patient via the MLC apertures or attenuation filter.

Turning now to FIG. 1, a diagram of a conventional multi-leaf collimator radiation treatment device 100 is shown. An electron beam 105 is generated by an electron accelerator 106. The electron accelerator 106 includes an electron gun 110, a wave guide 112, and an evacuated envelope or guide magnet 113. A triggering system 114 generates injector trigger signals and supplies them to an injector 115. Based on the trigger signals, the injector 115 generates injector pulses which are fed to the electron gun 110 in the accelerator 106 which results in the generation of electron beam 105.

The electron beam 105 is accelerated and guided by wave guide 112. A high frequency signal source (not shown) is also provided, which supplies RF signals for the generation of an electromagnetic field which is supplied to wave guide 112. The electrons injected by the injector 115 and emitted by the electron gun 110 are accelerated by the electromagnetic field in the wave guide 112 and exit at the end opposite to electron gun 110 in electron beam 105. The electron beam 105 then enters guide magnet 113 and from there is guided through window 117 along axis 118. After passing through a first scattering foil 119, the beam goes through an opening 120 of a shield block 122 and encounters a flattening filter 123. Next, the beam is sent through a measuring chamber 125 in which the dose is determined. If the scattering foil 119 is replaced by a target, the radiation beam is an X-ray beam. In this case, the flattening filter 123 may be absent.

Beam shielding device is provided in the path of beam 105, comprising a plurality of opposing plates 131 and 132, only two of which are illustrated for convenience. In one embodiment, other pairs of plates (not shown) are arranged perpendicular to plates 131 and 132. The plates 131 and 132 are moved with respect to axis 118 by a drive unit 134 to change the size and shape of the irradiated field. The drive unit 134 includes an electric motor which is coupled to the plates 131 and 132 and which is controlled by a motor controller 140. Position sensors 144 and 145 are also coupled to the plates 131 and 132, respectively for sensing their positions. As noted above, the plate arrangement may alternatively include a multi-leaf collimator having a plurality of radiation blocking leaves.

The motor controller 140 is coupled to a dosing unit 146 which includes a dosimetry controller and which is coupled to a central processing unit 148 for providing set values for the radiation beam for achieving given isodose curves. The output of the radiation beam is measured by a measuring chamber 125. In response to the deviation between the set values and the actual values, the dose control unit 146 supplies signals to a trigger system 114 which changes in a known manner the pulse repetition frequency so that the deviation between the set values and the actual values of the radiation beam output is minimized. In such a radiation device, the dose delivered is dependent upon movement of the collimator leaves 131 and 132.

The central processing unit 148 is typically programmed by the therapist according to the instructions of an oncologist which performs beam optimization so that the radiation treatment device carries out the prescribed radiation treatment while generally maximizing MU efficiency. Central processing unit 148 generally includes associated non-volatile memory (not shown). The delivery of the radiation treatment is generally input through a keyboard 151, or other suitable data entry device. The central processing unit 148 is further coupled to a dose control unit 146 that generates the desired values of radiation for controlling trigger system 114. The trigger system 114 then adapts the pulse radiation frequency and other parameters in a corresponding, conventional manner. The central processing unit 148 further includes a control unit 156 which controls execution of the software and the opening and closing of the collimator plates 131 and 132 to deliver radiation according to a desired intensity profile. A monitor 160 is also provided.

### SUMMARY

A method of determining a treatment plan for intensity modulated radiation treatment (IMRT) divides a three-dimensional volume of a patient into a grid of dose voxels. At least a portion of the dose voxels are designated to belong to at least one target or to at least one critical structure. An ionizing radiation dose as delivered by a plurality of beamlets each having a beamlet intensity is modeled. A non-linear convex voxel-based penalty function model is provided for optimizing a fluence map. The fluence map defines the beamlet intensities for each of the plurality of beamlets. The model is then solved based on defined clinical criteria for the target and the critical structure using an interior point algorithm with dense column handling to obtain a globally optimal fluence map.

The non-linear penalty functions can be selected from piece-wise linear functions, convex non-linear functions, and piece-wise non-linear convex functions. As used herein “dense column handling” is defined as the decomposition of a matrix into sparse and dense components to improve the efficiency of the interior point algorithm. The dense column handling preferably comprises Sherman Morrison Woodbury decomposition or Shur decomposition. The method can include the step of constraining the model with a dose-volume constraint to produce a constrained model. In this embodiment, the dose-volume constraint can bound a mean value of a tail of a differential dose-volume histogram (DVH) for a structure within the patient comprising a portion of the grid of dose voxels. In a preferred embodiment, the dose volume constraint comprises a conditional value at risk (CVaR) constraint. The CVaR constraint can includes an upper and lower bound constraints on the dose, or the mean dose, received by each of the voxels comprising a given target region within the patient. Use of a VCaR generally improves the quality of the solution at the cost of more computational time.

A system for delivering intensity modulated radiation treatment (IMRT), includes an inverse treatment planning system. The inverse treatment planning system includes a computing structure. The computing structure divides a three-dimensional volume of a patient into a grid of dose voxels, wherein at least a portion of the dose voxels are designated to belong to at least one target or to at least one critical structure, models an ionizing radiation dose as delivered by a plurality of beamlets each having a beamlet intensity, and implements a non-linear convex voxel-based penalty function model for optimizing a fluence map. The fluence map defines the beamlet intensities for each of the plurality of beamlets. The computing structure solves the model based on defined clinical criteria for the target and the critical structure using an interior point algorithm with dense column handling to obtain a globally optimal fluence map. A radiation source generates at least one radiation beam and structure is provided to generate the plurality of beamlets. A multi-leaf collimator is disposed between the radiation source and the patient. The collimator is communicably connected to the computing structure and has a plurality of leafs for modifying the plurality of beamlets to deliver the globally optimal fluence map to the patient.

### BRIEF DESCRIPTION OF THE DRAWINGS

A fuller understanding of the present invention and the features and benefits thereof will be accomplished upon review of the following detailed description together with the accompanying drawings, in which:

FIG. 1 is a diagram of a prior art radiation treatment device including a multi-leaf collimator.

FIG. 2(a) is an illustration of voxel-based nonlinear and convex penalty functions for a critical structure and a target, and PWL approximations; FIG. 2(b) is an illustration of a convex critical structure penalty function approximated by four line segments, and FIG. 2(c) is an illustration of a convex target penalty function approximated by eight line segments.

FIG. 3 is a schematic demonstration of the definition of a CVaR constraint imposed on a differential dose volume histogram (DVH).

FIG. 4(a) is DVHs of two optimized IMRT treatment plans, Plan 1 (red solid line), and Plan 2 (yellow solid line) for the same patient data with nearly identical target volumes covered by 70 Gy; FIG. 4(b) is an illustration of Plan 1 dose color-wash display shown on an axial slice through the superior aspect of the CTV; FIG. 4(c) is an illustration of Plan 2 dose color-wash display shown on the same axial slice through the superior aspect of the CTV.

FIG. 5 shows examples of a Dmax=45 Gy beam with dose calculated (labeled “calculated”) for a 1×1 cm² beam in water using a simple pencil beam model fit to RCF data at 2×2 cm² and 5×5 cm², an RCF measurement (labeled ‘measured’) for a 1×1 cm² beam and an isodose curve overlay for the calculated (solid lines) and the measured (dashed lines) distributions with curves at 40, 35, 30, 25, 20, 15, 10, 5 and 2 Gy. Both the measurement and the calculation were performed for a 0.1 mm sized voxel.

FIG. 6 is cumulative DVHs of tissue (solid line), PTV1 (dashed line), right parotid (dash-dot line), right submandibular (dotted line), PTV2 (solid line), from the solutions of the FMO problem employing the LP_{PWL} (thick lines) and LP_{PWL+CVaR} (thin lines) models.

FIG. 7(a) is an overlay of axial CT isodose curves (10, 26, 45, 50, 60, 70 Gy), dose colourwash, and structures (PTV1-blue, PTV2-orange, spinal cord-yellow, right submandibular gland-green, mandible-cyan) for the globally optimal solutions obtained by (a) the LP_{PWL} model and (b) the LP_{PWL+CVaR} model.

FIG. 8 is cumulative DVHs of tissue (solid line), PTV1 (dashed line), right parotid (dash-dot line), right submandibular (dotted line), PTV2 (solid line), from the solutions of the FMO problem employing the LP_{PWL} with an unspecified tissue resolution of 3 mm (UTR3; thick lines) and with an unspecified tissue resolution of 6 mm (UTR6; thin lines).

FIG. 9 is an overlay of three sets of DVHs for the original segments (thick lines), doubled number of segments (medium lines) and quadrupled number of segments (thin lines).

### DETAILED DESCRIPTION OF THE INVENTION

A method of determining a treatment plan for intensity modulated radiation treatment (IMRT) divides a three-dimensional volume of a patient into a grid of dose voxels. At least a portion of the dose voxels are designated to belong to at least one target or to at least one critical structure. An ionizing radiation dose as delivered by a plurality of beamlets each having a beamlet intensity is modeled. A non-linear convex voxel-based penalty function model is provided for optimizing a fluence map. The fluence map defines the beamlet intensities for each of the plurality of beamlets. The model is then solved based on defined clinical criteria for the target and the critical structure using an interior point algorithm with dense column handling to obtain a globally optimal fluence map. The non-linear convex voxel-based penalty functions can be selected from piece-wise linear functions, convex non-linear functions, and piece-wise non-linear convex functions.

The interior point method and variants thereof together with dense column handling is used because of its high efficiency and resulting generally short computational times. The interior point method is known in the art of optimization and is described in a book by Steven J. Wright entitled “Primal-Dual Interior-Point Methods” (SIAM Publications, 1997, ISBN 089871382X). This Wright paper is incorporated by reference into the application in its entirety and hereafter referred to as “Wright”. Wright also discloses dense column handling via Sherman Morrison Woodbury or Shur decomposition. Without dense column handling a practical sized model will not solve in a reasonable time (e.g. solution may take several days, or more).

Primal-dual algorithms have emerged as the most important and useful algorithms from the interior-point class. Wright discloses the major primal-dual algorithms for linear programming, including path-following algorithms (short- and long-step, predictor-corrector), potential-reduction algorithms, and infeasible-interior-point algorithms.

Interior point methods are used according to the invention to efficiently solve linear and quadratic programming problems to global optimality and unimodality. In principle, interior point methods can be applied to any purely convex problem, such as of order above 2 as well as fractional orders. Generally, the problem, is characterized by a matrix of the dose deposition coefficients (Dᵢⱼ). This matrix is very sparse since generally only about 0.1% of the matrix is nonzero. However, in terms of typical operations research problems the “density” of an optimization problem is an absolute value not a relative one. A “sparse” optimization problem typically has <5 nonzeros per column. This generally works out to be the number of voxels hit by a single beamlet and is between about 500 and 10,000 nonzeros. Thus “dense column handling” algorithms are implemented as described in Wright for dense columns with more than 3 nonzeros. Thus, in a preferred embodiment of the invention the convex model is solved to obtain a globally optimal solution using an interior point method, along with a “sparse” (in a relative sense) dose deposition coefficient matrix and dense column handling of the matrix.

Interior point methods solve convex programs by starting from a point lying in the interior of the feasible region. At each iteration, the objective is improved by moving to another point in the feasible region with better objective function. Variants of the primal-dual algorithm based either on reducing a logarithmic potential function or on explicitly following a central path (defined as the set of points at which the product of each primal-dual variable pair is identical) are described in literature. There are several variants of interior point methods but most are comparable. For example, ILOG CPLEX (ILOG, Inc. Mountain View, Calif.) uses the “log barrier” algorithm. This aspect is not important as other IPMs perform similarly.

In one embodiment, the convex objective function is first approximated using a piecewise linear (PWL) function to create a purely linear model before solving the same, again preferably using the interior point method. PWL approximation is not strictly required for all convex functions, such as linear or quadratic functions (see examples).

In the PWL embodiment, the invention thus provides a novel linear programming (LP) model for solving the fluence map optimization (FMO) problem in IMRT treatment planning. Prior to the invention, linear programming has found little use in radiation therapy due to the apparent limitations imposed by the linear form of the objective functions and the available constraints that can be used. However, the inventive approach to the FMO problem described herein overcomes limitations of LP by using piecewise linear (PWL) approximations of nonlinear convex penalty functions. Whether using the PWL approximation model or the original convex objective radiation model, the model can utilize a variety of constraints, provided only that the constraints can be expressed as a convex function. Generally suitable constraints include upper and lower bound constraints on the dose received by each voxel, as well as upper and lower bound constraints on the mean dose received by each target and adjacent critical structures (hereafter the “structures”).

Although the invention can be used without imposing a dose-volume constraint, in a preferred embodiment a dose value constraint is imposed. In this embodiment, a new type of dose-volume constraint that bounds the mean value of the tail of the differential dose volume histogram (DVH) of a target and structures is used. This type of constraint is referred to herein as a Conditional Value-at-Risk (CVaR) constraint and has only heretofore been used in financial applications. Value-at-Risk (VaR), a widely used financial performance measure, answers the question: what is the maximum loss with a specified confidence level? Generally, approaches to calculating VaR rely on linear approximation of risks and assume the joint normal (or log-normal) distribution of the underlying market parameters.

Conditional Value-at-Risk (CVaR), is also called Mean Excess Loss, Mean Shortfall, or Tail VaR. CVaR is a more consistent measure of risk as compared to VaR since it is sub-additive and convex. Moreover, CVaR can be optimized using linear programming (LP) and nonsmooth optimization algorithms, which allow handling portfolios with very large numbers of instruments and scenarios. Numerical experiments indicate that the minimization of CVaR also leads to near optimal solutions in VaR terms because CVaR is always greater than or equal to VaR.

Again regarding the PWL embodiment, the invention yields a robust FMO model as it retains linearity, and thereby unimodality (single solution) and efficient solvability of the problem. In addition, solution procedures for LP are able to recognize that an obtained solution is indeed optimal. In the remainder of this disclosure, unless specified otherwise, the inventive model will include a PWL objective function and CVaR constraints and be referred to generally as the LP_{PWL+CVaR}.

The main difference between the LP_{PWL+CVaR} model described herein and prior LP formulations of the FMO problem that have previously disclosed is the use of a PWL convex objective function. The PWL convex objective functions provide a high degree of modeling flexibility. A purely convex objective function is another distinction. The introduction of CVaR differential DVH constraints is a further element of distinction.

To formulate the FMO model according to the invention, structures are assumed to be irradiated using a predetermined and typically possibly large set of beams, each beam corresponding to a particular beam angle. For each beam angle, the beam aperture is decomposed or discretized into small beamlets, such as a typical of size 1×1 cm². A value is associated with each beamlet, and its value represents the intensity (or more correctly, fluence) of the corresponding beamlet. The central task in FMO is to find the optimal values of the beamlet intensities for each of the beamlets. A decision variables is defined representing the intensity of beamlet i by uᵢ, and denotes the decision vector of all beamlet intensities by [symbol] . Furthermore, the number of beamlets is denoted by N. The absorbed ionizing radiation dose received by each voxel is then expressed as a linear function of the beamlet intensities as follows:

$${D}_{js} ( \vec{u} ) = {\sum }_{i = 1}^{N} {D}_{ijs} {u}_{i} j = 1 , … , {n}_{V}^{s} , s = 1 , … , {n}_{S} ,$$

where Dᵢⱼₛ denotes the dose received by voxel j in structure s from beamlet i per unit fluence. It is noted that each voxel is uniquely identified by its structures and voxel number j in that structure. The number of targets is denoted by n_{T} and the total number of structure by n_{S}. Furthermore, each structure s is discretized into nᵥ^{s} voxels.

Certain structures may overlap. For instance, a target could invade a critical structure. However, in the inventive model a unique (i.e., dominant) structure can be associated with each voxel, based on a priority list of all structures, where targets usually have the highest priorities, followed by the critical structures, with the least important structure being unspecified tissue or skin. Note that this is not carried over to the treatment planning system, where dose and dose-volume criteria are appropriately evaluated for the complete structures. Beamlet dose models are inherently linear and are widely used to solve the FMO problem. The LP_{PWL+CVaR} model according to the invention can take the following form:
minimize the objective function:

$${\sum }_{s = 1}^{{n}_{S}} {\sum }_{j = 1}^{{n}_{V}^{s}} {F}_{s} ( {D}_{js} ( \vec{u} ) )$$

subject to the following constraints:
fluence nonnegativity constraints: [symbol] ≧0
upper and lower dose constraints: Lₛ≦ⱼₛ( [symbol] )≦Uₛ j=1, . . . , n_{V}^{s}, s=1, . . . , n_{S}
upper and lower mean dose constraints:

$${\underline{M}}_{s} \leq \frac{1}{{n}_{{V}_{s}}} {\sum }_{j = 1}^{{n}_{V}^{s}} {D}_{js} ( \vec{u} ) \leq {\overline{M}}_{s} s = 1 , … , {n}_{S}$$
upper CVaR constraints: \overline{φ}ₛ^{α′}( [symbol] )≦Uₛ^{α′} s=1, . . . , n_{S}
lower CVaR constraints: φₛ^{α}( [symbol] )≧Lₛ^{α} s=1, . . . , n_{T}.

The objective function preferably used is based on the sum of structure-dependent convex penalty functions Fₛ of the dose received by voxels in structure s. Using arbitrary convex penalty functions, a very general and flexible FMO model can be formulated. Possible penalty functions can include, but are not limited to, linear, quadratic, and higher order polynomial functions of dose.

Whereas doses in targets should be clustered around some prescription dose, it is assumed that lower doses in critical structures are always preferred to higher doses. This means that for voxels in these structures, the penalty function is always chosen to be one-sided. Thus, surpluses over a predetermined tolerance are penalized in the objective function.

The explicit form of the convex voxel-based penalty functions Fₛ is now described that is approximated in the basic model. The functions are selected to be asymmetric functions of dose. In particular, the penalty function for structure s, (Fₛ(Dⱼₛ), can be written as the sum of two terms:
Fₛ(Dⱼₛ)=Fₛ^{C}(Dⱼₛ)+Fₛ^{H}(Dⱼₛ),
where Dⱼₛ denotes the dose received by voxel j in structure s. The former term accounts for underdose penalty, and the latter term accounts for overdose penalty. The two terms are selected to be one-sided polynomials with respect to a so-called cold spot threshold dose Tₛ^{C} and a hot spot threshold dose Tₛ^{H}, respectively:
Fₛ^{C}(Dⱼₛ)=βₛ^{C}max{Tₛ^{C}−Dⱼₛ, 0}^{p}^{ₛ}^{^{C}} and Fₛ^{H}(Dⱼₛ)=βₛ^{H}max{Dⱼₛ−Tⱼₛ−Tₛ^{H},0}^{p}^{ₛ}^{^{H}},
where βₛ^{C} and βₛ^{H} are structure-dependent coefficients and pₛ^{C} and pₛ^{H} are powers of the piecewise polynomial penalty function for structure s. To ensure convexity, βₛ^{C},βₛ^{H}≧0 and pₛ^{C}, pₛ^{H}≧1 should be chosen.

When all penalty functions are PWL and convex, the problem can be formulated as a pure LP problem, which is referred to herein as the LP problem with PWL objective function: LP_{PWL}. Thus, PWL penalty functions can be written (assuming they have finitely many segments) as:

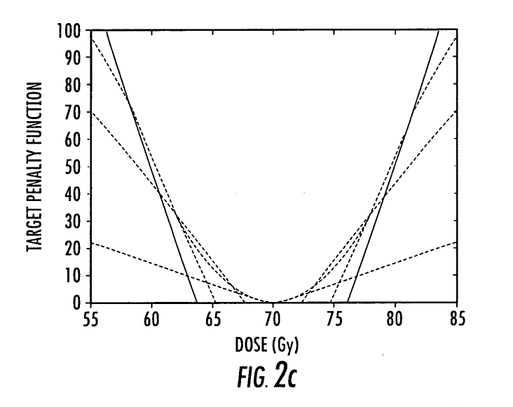

FIG. 2(a) is an illustration of voxel-based nonlinear and convex dose penalty functions for a critical structure and a target, and PWL approximations for target voxels with both cold spot and hot spot threshold doses equal to 70 Gy and critical structure voxels with a hot spot threshold dose of 30 Gy. FIG. 2(b) is an illustration of a convex critical structure penalty function approximated by four line segments, while FIG. 2(c) is an illustration of a convex target penalty function approximated by eight line segments.

It is noted that any convex PWL penalty function as the maximum of a number of linear penalty functions as follows:

$${F}_{s} ( {D}_{js} ) = {max}_{k = 1 , … , {m}_{s}} \{ {a}_{k}^{s} {D}_{js} + {b}_{k}^{s} \}$$

for suitably chosen slopes aₖ^{s} and intercepts bₖ^{s}, where mₛ denotes the number of linear segments defining the PWL function. It is this property that allows incorporation of an accurate approximation of a nonlinear convex objective function into a LP model.

Fluences are constrained to be physical, thus nonnegative. Hard upper and lower bounds on the voxel doses in structure s are denoted by Uₛ and Lₛ, respectively, and hard upper and lower bounds on the mean dose to structure s are denoted by \overline{M}₅ and Mₛ. The hard upper and lower dose and mean dose constraints are linear due to the linearity of Dⱼ( [symbol] ). The lower bound for dose to voxels in critical structures is usually set to 0.

Traditional dose-volume constraints control particular values of a structure DVH. As pointed out above, dose-volume constraints cannot be applied in a linear model without introducing integer-valued decision variables, leading to mixed integer linear programming (MILP). However, constraints of a different nature can be applied to the differential DVH. The problem of controlling the shape of the differential DVH resembles, to a large extent, a problem that has received much attention in the financial-engineering literature. This method is called the CVaR-approach, and has received widespread attention in recent years for formulating risk management constraints in financial applications. This methodology is preferably applied to solving the FMO problem. These constraints on the differential DVH generalize the mean dose constraints described above by constraining the mean dose received by subsets of voxels receiving the highest or lowest doses among all voxels in a given structure. More formally, the preferred constraints are of the following form:

(i) The average dose received by the subset of a target s of relative volume 1−α receiving the lowest amount of dose, called the lower α-CVaR and denoted by φₛ^{α}( [symbol] ), must be at least equal to Lₛ^{α}.

(ii) The average dose received by the subset of a structure s of relative volume 1-α′ receiving the highest amount of dose, called the upper α′-CVaR and denoted by \overline{φ}ₛ^{α′}( [symbol] ), may be no more than Uₛ^{α′}.

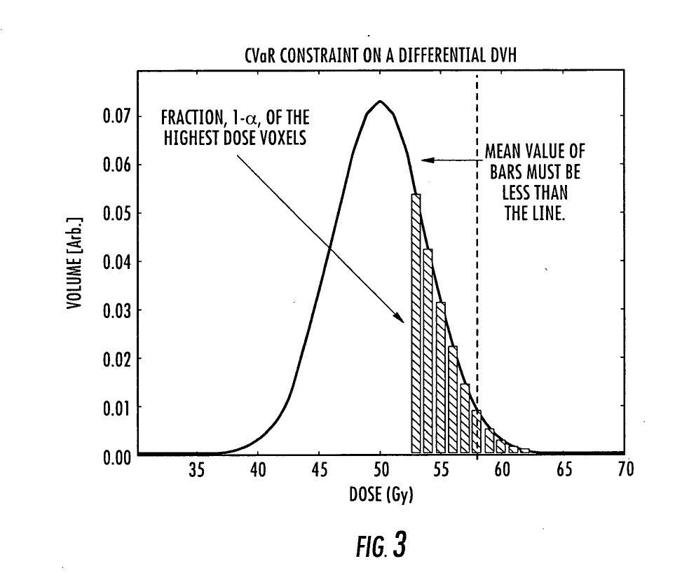

FIG. 3 illustrates an example of an upper CVaR constraint applied to a particular differential DVH. The CVaR constraints reflect the fact that the upper and lower bounds Uₛ^{α′} and Lₛ^{α} may be violated by some subset of the voxels in structure s. Note that a CVaR constraint does not force a fraction of the voxels to violate a bound. This approach has two major advantages. While this technique does not directly impose a traditional dose-volume constraint, it is intuitive from a treatment-plan quality point of view, as it simultaneously limits the fraction of voxels that violate a soft bound, as well as the mean dose of all voxels violating that bound. From a computational point of view, CVaR constraints have the attractive property that they are convex and can be incorporated into the inventive model while retaining linearity. In case a target has invaded a critical structure to which we wish to apply an upper CVaR constraint and a sufficient volume of the structure exists outside the target to spare, we choose to reformulate this CVaR constraint to only apply to the part of the critical structure outside the target, i.e., the set of voxels in the critical structure for which this structure is dominant, by suitably redefining the fraction α.

Spatial dependencies can be incorporated into the FMO objective function. Reasons for incorporating spatial dependencies and the limitations of dose-volume constraint based plan evaluation have been known since the inception of the DVH. The traditional objective of three-dimensional conformal radiation treatment (3DCRT) planning (we exclude IMRT in this definition of 3DCRT), a homogeneous target dose distribution, is often not achievable in clinical settings due to the tolerance limits of adjacent critical organs. Thus, the target DVH assumes the shape of a sigmoidal curve rather than a step function, indicating the presence of hot and cold spot inhomogeneities in the dose distribution. The merit of a 3DCRT plan is typically assessed with dose-volume constraints derived from a DVH. Although a DVH provides no spatial information regarding the location of hot and cold spots, this tool provides an adequate assessment of planning target volume (PTV) coverage because of the uniform fields employed in 3DCRT. One does not need spatial correlations for 3DCRT as cold spots exist on the periphery of the target volume due to a failure to accurately conform to the shape of the target, and hot spots exist in locations that can be predicted by the beam and patient geometry. Hot spots are often acceptable due to their limited magnitude, which can often be kept under 5% of the prescription dose for a well-designed plan.

In contrast to 3DCRT, the optimal fluence map produced for IMRT delivers multiple small subfields with various intensities to the target, and this IMRT delivery may allow for the spreading of hot and cold spots throughout the target and critical-structure volumes and render DVH- or dose-volume constraint based evaluation of IMRT plans insufficient. A common feature of all FMO models employed thus far is that they are insensitive to spatial characteristics of the dose distribution. In other words, these optimization algorithms implicitly assume that all voxels within a given target volume have equal clinical importance. Given that a less-than-perfect target coverage will in some cases be produced by an optimization, a valid concern rises over the location of hot and cold spots generated by optimizing plans according to current FMO models.

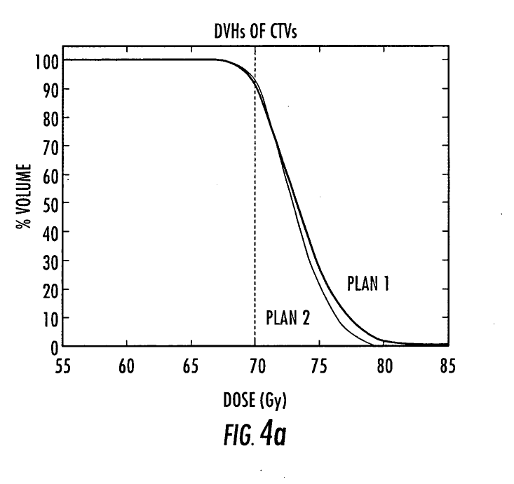

To illustrate this situation with a concrete clinical example, FIG. 4(a) shows the DVHs from two optimized IMRT treatment plans. Arbitrarily labeled Plan 1 and Plan 2, the two plans demonstrate a nearly equivalent degree of clinical target volume (CTV) coverage by 70 Gy. Moreover, both plans would be considered clinically acceptable if they were solely evaluated by virtue of their DVH-based target coverage, with a slight preference for Plan 2 which provides a decreased hot spot and slightly improved coverage. The slight preference for Plan 2 vanishes when an axial view of Plan 1 is compared to that of Plan 2 at the same level in the superior aspect of the CTV as shown in FIGS. 4(b) and 4(c). It is apparent that Plan 2 results in a significant cold spot at the center of the CTV, which happens to contain gross disease that is radiographically evident but not segmented in the plan. When the two plans were compared at the inferior aspect of the CTV (not shown), Plan 1 introduced an underdosed region at the periphery of the CTV, which was not evident in Plan 2. This example illustrates how the assumption of equal merit for different target volume subregions is intuitively unsatisfactory.

A basic underlying assumption made by all FMO models is that plans with equivalent dose-volume constraints have equivalent clinical quality. Stated another way, this assumption implies that subregions of a clinical target have equal importance regardless of their location. This limitation in the use of dose-volume information was well understood early on in the development of conformal radiotherapy; however, early caveats appear to have been lost in the current practice of treatment plan optimization and evaluation. While one might be attempted to apply “biological” models to remedy this problem, it follows that any quantity that is simply derived from the spatially independent dose-volume information, such as tumor control probabilities (TCP), normal tissue complication probabilities (NTCP), or EUD, inherits the lack of spatial information. In other words, if TCP, NTCP, and EUD can be computed without spatial information, how can they solve this problem?

Previous studies have proposed to either incorporate functional or positional information into dose-volume based plan criteria to improve the ability to evaluate the clinical merit of a treatment plan. However, these techniques have not been widely employed and either require physiological and multimodality imaging data or have been limited to image-based spatial coordinates rather than anatomically based coordinates. Accepting that existing biological models are too crude to form a basis for formulating a FMO model, some “clinical common sense” can be instilled into our approach to the problem by making subregions of a target more important as distances from the target surface increase.

Even with a highly efficient and efficacious method for finding globally optimal solutions to the FMO problem, it has to be accepted that for some cases not all clinical plan criteria will be satisfied. While the preliminary data demonstrates that an objective function can be developed to satisfy all the clinical criteria in a large fraction of the cases, a sound strategy must be in place to deal with those cases that fail. A very appealing method for dealing with this problem that has been put forward is the multi-criteria optimization approach discussed above. Populating the Pareto-efficient frontier with thousands of solutions makes it quite computationally inefficient. Even with our LP optimization times of less than three minutes and the parallel computing grid proposed in this study, many hours of computation time would be required for a single run. The interest in this approach will likely grow in the future when increases in computing power can make it feasible for the LP_{PWL+CVaR} model. As a more efficient alternative, the treatment planner can be allowed the option of using a GUI that allows for the definition and redefinition of regions of increased importance while reviewing a failed treatment plan. Then, rapid re-optimization techniques can be applied to efficiently guide the treatment plan to a satisfactory tradeoff. To model the increased or decreased importance of certain regions, spatial dependencies can be integrated into the objective function. In the LP_{PWL+CVaR} model, this can be accomplished by introducing a weighting factor for each voxel, yielding the following modified objective function:

$${\sum }_{s = 1}^{{n}_{S}} {\sum }_{j = 1}^{{n}_{V}^{s}} ( {σ}_{js}^{C} {F}_{s}^{C} ( {D}_{js} ) + {σ}_{js}^{H} {F}_{s}^{H} ( {D}_{js} ) ) ,$$

where σⱼₛ^{C} is a weighting factor associated with cold spots involving voxel j in structure s, and σⱼₛ^{H} is a weighting factor associated with hot spots involving that voxel. Scaling the independent voxel terms of the objective function has no effect on the convexity or linearity of the problem and does not increase the size of the problem. We envision two methods for determining the weighting factors. The first method is referred to as the anatomical method and would use the surfaces of structures to provide a metric for importance. This will allow the treatment planner to “make statements to” the objective function like: “cold spots are preferable if they are on the periphery of a target” or “hot spots are preferable if they are further from a critical structure or closer to a target”. The second method requires the treatment plan reviewer to interactively segment regions of the patient that should be assigned a higher or lower priority. Given the efficiency of the optimization technique described herein which allows plan re-optimizations in less than three minutes, it is believed that this approach will eventually translate well into a clinical setting as can keep the attention of a treatment planner while they iteratively add or remove spatial dependencies into the FMO model to guide or control the clinical tradeoffs.

The invention also provides a method for restoring the traditional dose delivered per fraction to IMRT. The majority of conformal external-beam radiotherapy has been developed using a narrow range of dose delivered per fraction. This range has typically been limited to 1.8-2.0 Gy per fraction in a daily fractionated scheme, with limited studies of twice-daily hypofractionated schemes having 1.1-1.5 Gy per fraction and daily hyperfractionated schemes having 2.2-2.5 Gy per fraction. Treatments were typically carried out using independent conformal plans that were manually designed for each target dose level in a plan delivered at a single dose per fraction. As the cumulative dose delivered reached each successive target level, new portals were introduced to “cone-down” on higher dose targets. With the advent of IMRT, it has become a practical impossibility to reproduce this constant dose-per-fraction delivery technique with a single optimized plan. This is due to the fact that it has become a standard of practice to only solve the FMO problem for a single set of fluence maps (with a single fluence map for each beam) at a time. Currently, all commercial and most research IMRT treatment planning follow this practice of optimizing a single set of fluence maps.

If a single plan is optimized to deliver dose to multiple target-dose levels, then the dose per fraction delivered to each target must change in the ratio of a given dose level to the maximum dose level. There are essentially two approaches to dealing with the different doses per fraction that are delivered to targets when a single set of fluence maps is employed. The first is to set the highest target-dose level to the traditional dose per fraction and allow lower-dose targets to be treated at lower doses per fraction. The concern with this approach is that the lower-dose targets may not receive sufficient cell kill, so the total doses are increased for these targets using a radiobiological model. The second approach is to set the lowest target-dose level to the traditional dose per fraction and allow higher-dose targets to be treated at higher doses per fraction. The concern with this approach is that the higher-dose targets receive much higher biologically effective doses that could result in an increase in iatrogenic effects. Several research groups have suggested that delivering different doses per fraction to different targets can have clinical benefits, and some single institution trials have been initiated to study this hypothesis. The inventive system can provide such solutions. To avoid having different doses delivered per fraction to different target-dose levels, multiple plans must be produced independently. In this case, the solution is not optimal. This is also possibly dangerous requiring extra care in reviewing the cumulative plan as structure sparing is not ensured for the cumulative dose delivered and, as discussed in paragraph [00046] above, there is no spatial information in a DVH. The model is preferably extended to allow for the simultaneous optimization of multiple sets of fluence maps for multiple target-dose levels. This will allow IMRT practitioners to reproduce the dose fractionation schemes that were developed with 3DCRT and take advantage of the clinical experience developed with these techniques.

In order to optimize multiple sets of fluence maps (one for each target or prescription-dose level) simultaneously, decision variables representing the intensity of beamlet i in fluence-map set f are defined as u_{if}, and the decision vector of all beamlet intensities in fluence-map set f by [symbol] _{f}. The absorbed ionizing radiation dose received by each voxel in fluence-map set f are expressed as a linear function of the beamlet intensities [symbol] _{f} as follows, analogous to the expression for Dⱼₛ:

$${D}_{js}^{f} ( {\vec{u}}_{f} ) = {\sum }_{i = 1}^{N} {D}_{ijs} {u}_{if} j = 1 , … , {n}_{V}^{s} , s = 1 , … , {n}_{S} , f = 1 , … , {n}_{T} .$$

For voxels in critical structures, the relevant dose is the cumulative dose received in all fluence-map sets. However, the situation is more complicated for targets. For notational convenience, it is assumed in this section that the targets are ordered in increasing order of prescription dose and each target has a corresponding unique prescription dose. This means that in fluence map set f there is interest in treating all targets with a prescription dose larger than or equal to T_{f}. However, an underdosing of voxels in these targets in earlier fluence-map sets cannot be compensated for in the current fluence-map set, as this would allow the optimization to change the dose delivered per fraction. Therefore, an artificial cumulative dose received by target voxels is defined in the first f fluence-map sets (\tilde{D}ⱼₛ^{f}( [symbol] _{f})) as the sum of the prescription dose for the preceding fluence-map set (T_{f-1}) and the dose received in fluence-map set f (Dⱼₛ^{f}( [symbol] _{f})). Hence,
\tilde{D}ⱼₛ^{f}( [symbol] _{f})=T_{f-1}+Dⱼₛ^{f}( [symbol] _{f}) j=1, . . . , n_{V}^{s}, s=1, . . . , n_{T}, f=1, . . . , n_{T}.
In the objective function, the artificial cumulative doses for target voxels in each fluence-map set are penalized according to the appropriate target-dependent penalty function. Each fluence-map set will only see the target voxels that are included in its dose level. In addition, the true cumulative dose received by target voxels will be penalized as unspecified tissue for each fluence-map set that does not see it. Finally, the true cumulative dose received by voxels in all critical structures are penalized as in the single FMO model according to the invention. This leads to the following objective function:

$$minimize {\sum }_{f = 1}^{{n}_{T}} {\sum }_{s = 1}^{{n}_{S}} {\sum }_{j = 1}^{{n}_{V}^{s}} ( {\sum }_{ℓ = 1}^{f} {F}_{f} ( {\tilde{D}}_{js}^{ℓ} ( {\vec{u}}_{ℓ} ) ) + ( {n}_{T} - f ) {F}_{{n}_{S}} ( {\sum }_{f = 1}^{{n}_{T}} {D}_{js}^{f} ( {\vec{u}}_{f} ) ) ) + {n}_{T} {\sum }_{s = {n}_{T} + 1}^{{n}_{S}} {\sum }_{j = 1}^{{n}_{V}^{s}} {F}_{s} ( {\sum }_{f = 1}^{{n}_{T}} {D}_{js}^{f} ( {\vec{u}}_{f} ) ) .$$

As shown below, preliminary data is presented which indicates that computation time increases linearly with the number target dose levels, and that treatment plan quality can largely be preserved.

FMO may be viewed as a massive resource allocation problem, where one must decide which beamlets should be applied and to what extent. In principle, optimization should be applied to all aspects of IMRT treatment planning. This holistic optimization theoretically includes: selection of beam number, selection of beam orientation, selection of beam quality, selection of the fluence distributions and their discretization into deliverable sequences. The benefit of this approach is that all of the goals and constraints of the problem are considered simultaneously, possibly leading to a true globally optimal treatment plan. Of course, while formulating such a model could be considered, it has an astronomical dimensionality and cannot be practically solved by any currently available optimization algorithm. Therefore, in practice, this optimization problem is typically broken into three subproblems that can be solved to optimality or heuristically. When solving the IMRT treatment planning problem, the optimizations or decisions to be made are typically divided as such: (i) determine the number and orientations at which radiation beams will be delivered; (ii) determine the fluence map(s) for each radiation beam selected in (i); and (iii) determine a method of discretization of the fluence map produced in (ii) to produce a deliverable treatment plan. While subproblems (ii) and (iii) have typically required optimization for a high quality result, beam orientations from subproblem (i) are often selected ad-hoc in concordance with previous conformal therapy practices.

The literature contains many examples of studies that only deal with a single subproblem of the general IMRT optimization problem. In all commercially available systems for treatment planning, these subproblems are considered separately as well. There is, however, a growing realization that much better solutions may be obtained by using a more formal approach of integrating these optimization problems. Recently, work on the integration of pairs of these subproblems has begun, at least in some limited way. Examples include integration of (i) and (ii), (ii) and (iii) and even a limited integration of (i) and (iii) even into single models. These integrated approaches can provide the most desirable outcome as they allow one to perform a single optimization that considers all of the aspects of both subproblems simultaneously. When subproblems are considered independently and sequentially, the solutions are often not optimal with respect to all of the goals and constraints of both problems. The task of integrating requires very efficient algorithms for solving the subproblems to combat the increased dimensionality of the problem.

Beam-orientation optimization (BOO) presents a significant mathematical challenge as the introduction of beam-orientation degrees of freedom into a FMO problem results in either a nonconvex objective function, even if the objective function was convex for the original FMO problem or a problem with disjoint feasible solutions spaces. In either case, this leads to the existence of local minima and a hard problem. Thus, integration of the BOO and FMO problems have made use of heuristics based on conventional conformal radiation therapy. The approaches that have been proposed to date for optimizing the beam angles can be categorized into two broad classes. In the first approach, each candidate beam angle is given a numerical rating based on its effect on the targets and critical structures. Then, a prespecified number of beams with the best numerical ratings are then selected. The second approach, studied by Pugachev et al., is a local-search based approach. Initially, a given number of beam positions are selected, and corresponding beamlet intensities are determined. Then, one or more beam positions are changed, new beamlet intensities are determined, and the impact of this change is assessed. Generally, if the change improves the treatment plan, the change is made; otherwise another change is considered. This process is then repeated iteratively. Variants of this approach as part of a simulated annealing or genetic algorithm framework have also been implemented and studied. While many of these studies have found that optimizing beam angles can significantly improve the resulting treatment plan, the above approaches are somewhat limited as it is well known that the true influences of a set of gantry angles on the final dose distribution is not known until optimization of the beam intensity profile is performed. Moreover, owing to the heuristic nature of the algorithms, the current methodologies cannot provide a fair comparison between plans with different numbers of beams.

Due to the efficiency and efficacy of the LP model described herein for solving the FMO problem, solving a sizeable subset of the BOO problem exactly by discretizing the search space into convex sub problems can be considered. Such exhaustive searches have been proposed for conformal beam optimizations. However, the invention is the first disclosure to propose it for IMRT FMO. In the approach described herein, solving the integrated BOO and FMO problem for discrete numbers of limited beam set orientations is considered. The beam orientations that give the best treatment plan are then selected. The LP_{PWL+CVaR} model provides globally optimal solutions for each convex subproblem and the best solution of these is then the globally optimal solution for the full nonconvex subproblem. This includes coplanar equispaced BOO, coplanar non-equispaced BOO for small beam number (5-7 beams), and BOO for the addition of a non-coplanar beam to a predefined set of beams. Thus, the invention provides an exact algorithm for solving this subset of the BOO problem to provide an optimization benchmark. This benchmark will help unequivocally answer basic delivery questions like: “How many equispaced (and non-parallel opposed) coplanar beams should be used for treatment?”; “Should the angle of an equispaced set of beams be optimized?”; “What is the benefit of a tomotherapy approach?”; “What is the marginal benefit of adding another couple of beams to a plan?”; and “What if one of those beams is non-coplanar?”. Of course, the method can be applied to plans that do not satisfy all evaluation criteria in SA1 and answers to all but the first question for this entire subpopulation can be provided. The last question can be answered in only a few anecdotal cases due to the vastness of this problem, but even a limited number of exact solutions will provide a valuable benchmark for further development of heuristic methods like those described above.

Multi-criteria optimization can also be used with the invention. Multi-criteria optimization relates to trade-offs between different criteria in a given model. For example, assume that a set of structure based coefficients for a given convex model lead to a particular solution. However, for some individual cases there is a greater desire for improved or decreased sparing or coverage as compared to the particular solution generated by the model. To achieve multi-criteria optimization the coefficients or weights of the penalty functions which represent the criteria for the particular solution can be changed. Different sets of weights lead to different solutions. Having a convex model allows efficient mapping out of so-called Pareto Efficient solutions. Moreover, as described in a paper entitled “A Unifying Framework for Multi-Criteria Fluence Map Optimization Models” by Romeijn et al. (2004 Phys. Med. Biol. 49 1991-2013), “competing” models can be transformed into a model according to the invention when the competing models are viewed as multi-criteria optimization problems.

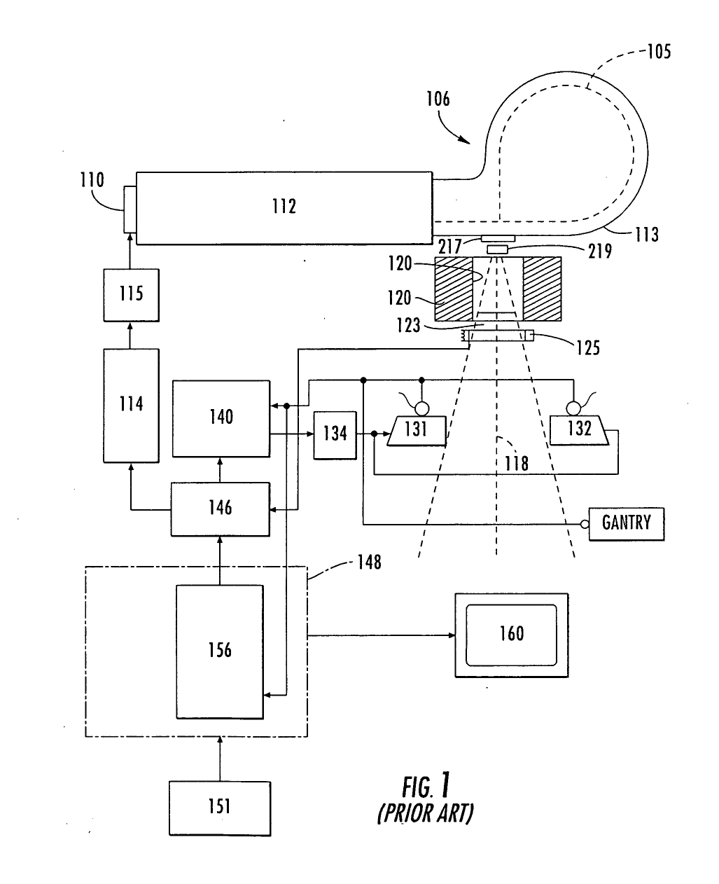

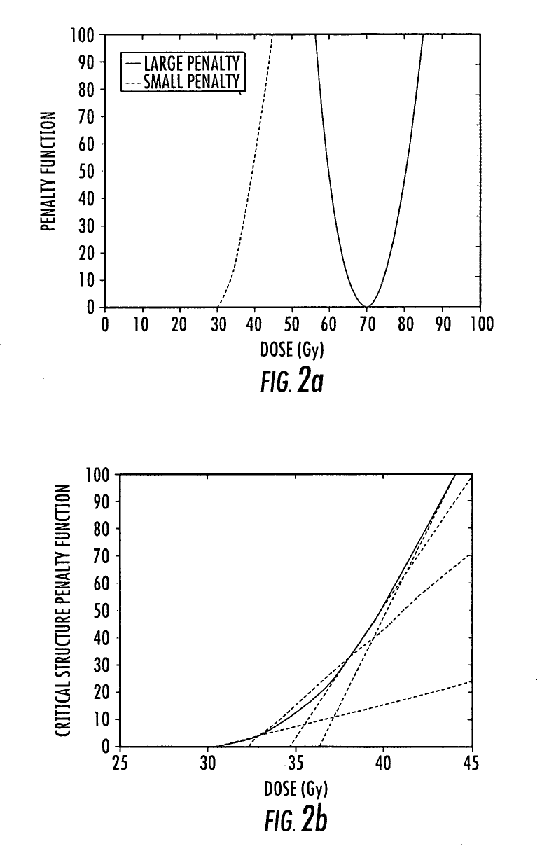

The invention is generally applicable intensity modulated radiation treatment (IMRT) systems, and can be applied to new systems as well to existing systems. For example, the invention can be applied to the radiation treatment device including multi-leaf collimator shown in FIG. 1. Referring again to FIG. 1, algorithms according to the invention for obtaining a globally optimum fluence map are stored in non-volatile memory or read in from another medium (e.g. disk). Control unit 156 of central processing unit 148 controls execution of stored software including algorithms according to the invention for obtaining a globally optimum fluence map, which opens and closes collimator plates 131 and 132 to deliver radiation according to the globally optimum fluence map determined according to methods described herein. IMRT according to the invention can be performed either while the beam is on, which is referred to as dynamic MLC or DMLC delivery, or by turning the beam off while the leaves move to their next position, which is referred to as segmented MLC or SMLC delivery.

### EXAMPLES

The present invention is further illustrated by the following specific Examples, which should not be construed as limiting the scope or content of the invention in any way.

To test linear programming models according to the invention, three-dimensional image-based treatment-planning data for head-and neck cancer patients were generated on an in-house treatment-planning system referred to herein as the University of Florida optimized radiation therapy (UFORT) treatment planning decision support system (TPDSS). The UFORT TPDSS was developed using a commercial technical programming language (Matlab, Mathworks Inc.). This system was designed to accept DICOM communications of treatment planning image, structure and plan data from a commercial treatment planning simulation software (VoxelQ, Philips Medical Systems) in the UF clinic. The system anonymized the patient data for research purposes and converted the data to an internal data format.

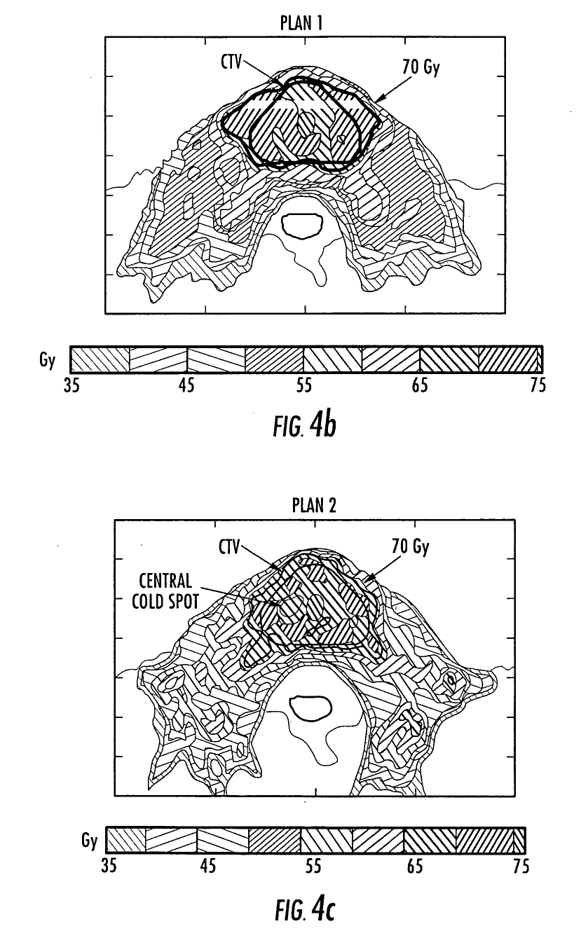

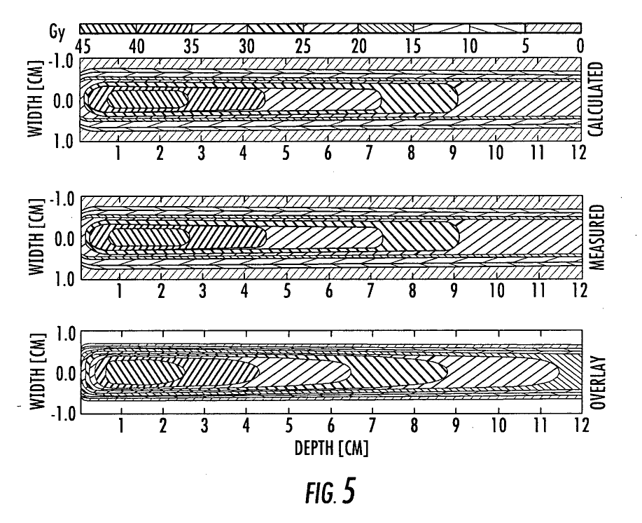

Users followed four steps to execute a FMO for treatment planning: (1) the isocenter to use for dose calculation was identified; (2) the critical organ and target-structure names were associated with unique structures on a list of expected structures; (3) prescription doses for targets were defined; and (4) the number and angles of beams were specified. Margins for penumbra were automatically generated for the union of the targets in each case, and asymmetric secondary jaw settings were determined. The beam apertures were then discretized into 1×1 cm² beamlets. An isotropic 3×3×3 mm³ dose voxel grid was generated, and the centroid of each dose voxel was given a unique label and tested for membership in all of the structures and recorded. To compute dose, a simple empirical beamlet model was employed that was fitted to radiochromic film data of 1×1 cm beamlets formed by secondary divergent jaw collimators and measured using a previously reported methodology (Validation of a precision radiochromic film dosimetry system for quantitative two-dimensional imaging of acute exposure dose distributions, Med. Phys. 27: 2462-75, Dempsey et al.). Dose due to leakage through the collimators was subtracted from the tails of the small beam data used for the fit as this information makes the Dᵢⱼ data unnecessarily dense and does not accurately represent the leakage produced by the actual sequenced delivery. An example of the accuracy obtainable with this model is presented in FIG. 5. The inventive method required between 0.7 and 1 s of computational time per beamlet on a 2.8 GHz Pentium 4 computer. The data generated by this method were written to a double-precision floating-point sparse matrix of Dᵢⱼ values.

A computer program was developed in C++ to interface with an industrial LP solver (CPLEX 8, ILOG Inc.). This interface program reads in the model data from the TPDSS and prepares it in a format (Concert Technologies, ILOG) known to the LP solver. Then the model is solved using the solver's implementation of the barrier interior-point method (Wright 1997). Once the model is solved to optimality, the optimal intensity vector, [symbol] * is written to a file for the UFORT system. The optimal intensities were discretized for each beam angle to a user selectable percentage (in this case 5% levels) in preparation for leaf sequencing. The resulting plan dose distribution and histograms were computed by summing the Dᵢⱼ weighted by the discretized intensities as in

$${D}_{js} ( \vec{u} ) = {\sum }_{i = 1}^{N} {D}_{ijs} {u}_{i} j = 1 , … , {n}_{V}^{s} , s = 1 , … , {n}_{S} , .$$

Leaf-transmission leakage intensities were estimated at 1.7% for otherwise zero intensity bixels. The plans were then reviewed using a graphic user interface that allows exploration of structure, DVH and dose data.

The form of the convex voxel-based penalty functions Fₛ(Δⱼ) that we approximated for this example are now discussed. For each structure sεS, we specify a lower bound Ls and an upper bound Us (where, for critical structures sεS², we have Ls=0), deviations from which are heavily penalized. In addition, each structure also has a threshold dose Ts, and deviations from this value are penalized using an asymmetric piecewise polynomial function:

$${F}_{s} ( {Δ}_{j} ) = \{ \begin{matrix}{{β}_{s} ( {T}_{s} - {L}_{s} )}^{{n}_{s}} + {\overline{β}}_{s} ( {L}_{s} - {Δ}_{j} ) & if & {Δ}_{j} < {L}_{s} \\ {β}_{s} {max ( {T}_{s} - {Δ}_{j} , 0 )}^{{n}_{s}} + {γ}_{s} {max ( {Δ}_{j} - {T}_{s} , 0 )}^{{m}_{s}} & if & {L}_{s} ⩽ {Δ}_{j} ⩽ {U}_{s} \\ {{γ}_{s} ( {U}_{s} - {T}_{s} )}^{{m}_{s}} + {\overline{γ}}_{s} ( {Δ}_{j} - {L}_{s} ) & if & {Δ}_{j} > {U}_{s}\end{matrix}$$

where βₛ, \overline{β}ₛ, γₛ and \overline{γ}ₛ are structure-dependent coefficients, and ns and ms are powers of the piecewise polynomial penalty function. The parameters βs and ns are associated with underdosing a voxel, while the parameters γs and ms are associated with overdosing a voxel. To ensure convexity, note that βₛ, γₛ≧0 and nₛ, mₛ≧1, should be chosen. The coefficients penalize deviations from the lower and upper bounds, and should be chosen large enough to ensure convexity of the penalty function. Note that, for critical structures sεS², Ls=βs=0 is always chosen.

When CVaR constraints are added, deviations of the corresponding bounds are allowed at a very high penalty. To achieve this, the CVaR lower and upper bound constraints are modified by introducing additional artificial variables φₛ^{α} and \overline{φ}ₛ^{α} as follows:

$${\underline{ζ}}_{s}^{α} + \frac{1}{( 1 - α ) \vert {V}_{s} \vert} {\sum }_{j ∈ {V}_{s}} {\underline{w}}_{j}^{α} = {\underline{ϕ}}_{s}^{α} α ∈ {\underline{A}}_{s} s ∈ {S}^{1}$$

$${\overline{ζ}}_{s}^{α} - \frac{1}{( 1 - α ) \vert {V}_{s} \vert} {\sum }_{j ∈ {V}_{s}} {\underline{w}}_{j}^{α} = {\overline{ϕ}}_{s}^{α} α ∈ {\overline{A}}_{s} s ∈ S$$

$${\underline{ϕ}}_{s}^{α} free α ∈ {\underline{A}}_{s} s ∈ {S}^{1}$$

$${\overline{ϕ}}_{s}^{α} free α ∈ {\overline{A}}_{s} s ∈ S .$$

The following terms are added to the objective function:

$${\sum }_{s ∈ {S}^{1}} {\sum }_{α ∈ {\underline{A}}_{s}} {\underline{G}}_{s}^{α} ( {\underline{ϕ}}_{s}^{α} ) + {\sum }_{s ∈ S} {\sum }_{α ∈ {\overline{A}}_{s}} {\overline{G}}_{s}^{α} ( {\overline{ϕ}}_{s}^{α} )$$

A novel linear programming approach to fluence map optimization for IMRT where
where
   Gₛ^{α}(φₛ^{α})=ηₛ^{α}max(0, Lₛ^{α}−φₛ^{α})
   \overline{G}ₛ^{α}(\overline{φ}ₛ^{α})=\overline{η}ₛ^{α}max(0,φₛ^{α}−Uₛ^{α})
   and ηₛ^{α}·\overline{η}ₛ^{α} are nonnegative slope parameters. Similar to the linear reformulation of the voxel based penalty functions, artificial variables can be used to incorporate these additional terms into the LP model.

Finally, for defining a dominant structure for each voxel a priority list has been used in which targets have the highest priorities, followed by the critical structures, with the least important structure being unspecified tissue or skin. It is noted that when the bound constraints are relaxed and include a penalty for violating them, the use of dominant structures is not required, since any inconsistency can be sorted out by the optimization. However, this approach has not been taken.

The clinical goals of optimization were to simultaneously cover 95% or more of two planning target volumes, PTV1 and PTV2, to doses of 70 and 50 Gy, with the minimum dose bounded within 7% of the prescription dose for both targets, and the maximum dose bounded within 10% of the prescription dose for PTV1 only. It was necessary to allow hot spots in PTV2 to obtain adequate coverage of PTV1, as PTV1 is a subset of PTV2. The following requirements were also defined: at least one of four salivary glands should have 50% or less of its volume covered by 30 Gy or higher (Intensity-modulated radiation therapy in head and neck cancers: the Mallinckrodt experience, Int. J. Cancer 90: 92-103, Chao et al, 2000); the spinal cord should have 99% or more of its volume covered by less than 45 Gy; the brainstem should have 99% of its volume covered by less than 50 Gy; the unspecified tissue (often referred to as ‘skin’ or ‘tissue’) should have 97% of its volume covered by less than 50 Gy.

A single case was first examined with seven equispaced beams having International Electrotechnical Commission (IEC) gantry angles of 0, 51, 103, 154, 206, 257 and 309. The UFORT system generated 1182 beamlets to adequately cover the targets from the seven beam angles, and the 3 mm isotropic voxel grid resulted in 206,152 voxels and generated 1,876,965 nonzero Dij values in a sparse matrix of size 1182 by 206,152 (density: 0.77%) that were output by the planning system.

The parameters of the LP_{PWL} model were determined by manual adjustment and are shown in Table 1 below. Similar to Tsien et al (Intensity-modulated radiation therapy (IMRT) for locally advanced paranasal sinus tumors: incorporating clinical decisions in the optimization process, Int. J. Radiat. Oncol. Biol. Phys. 55: 776-84, 2003), it was found that high powers of dose difference lead to excellent results. It was chosen to approximate the piecewise polynomial penalty functions for the targets by two PWL segments for underdosing and four segments for overdosing, and for the critical structures by three segments for overdosing. (not counting the segments penalizing violations of the bounds). Ipsilateral (left) salivary glands were not spared due to their proximity to PTV1.

TABLE 1
Values of the coefficients of the voxel-based penalty functions
that were used in solving the illustrated case.

| Structure (s) | Tₛ | Lₛ | βₛ | nₛ | \overline{β}ₛ | Uₛ | γₛ | mₛ | \overline{γ}ₛ |
|---|---|---|---|---|---|---|---|---|---|
| PTV1 | 72.5 | 69.5 | 20 | 12 | 10¹¹ | 75.5 | 20 | 6 | 10¹⁰ |
| PTV2 | 52 | 49.5 | 7 | 12 | 10⁹ | 55.5 | 7 | 6 | 10⁸ |
| Right parotid | 0 | 0 | 0 | — | — | 75.5 | 500 | 4 | 10¹¹ |
| Right | 0 | 0 | 0 | — | — | 75.5 | 5500 | 4 | 10¹¹ |
| submandibular | | | | | | | | | |
| Tissue | 28 | 0 | 0 | — | — | 75.5 | 300 | 5 | 10¹¹ |
| Spinal cord | 40.5 | 0 | 0 | — | — | 45 | 0.5 | 2 | 10¹⁰ |
| Brainstem | 45 | 0 | 0 | — | — | 50 | 0.6 | 2 | 10¹⁰ |
| Mandible | 70 | 0 | 0 | — | — | 77 | 0.3 | 2 | 10⁶ |

To demonstrate the utility of the CVaR constraints, an instance of the LP_{PWL+CVaR} model was formulated that penalized both contralateral (right) salivary glands as tissue (i.e. no attempt to spare the glands using the objective function), while adding upper CVaR constraints to achieve sparing of these structures. In addition, lower and upper CVaR constraints were added on both targets. The corresponding coefficients were again determined by manual adjustment and are shown in Table 2 below.

TABLE 2
Values of the coefficients corresponding to the CVaR constraints
that were used in solving the illustrated case.

| | Lower CVaR-constraints | | | Upper CVaR-constraints | | |
|---|---|---|---|---|---|---|
| Structure (s) | α | Lₛ^{α} | ηₛ^{u} | α | Uₛ^{α} | \overline{η}ₛ^{α} |
| PTV1 | 0.90 | 68 | 10¹² | 0.99 | 75 | 10¹¹ |
| PTV2 | 0.95 | 48 | 10¹² | 0.95 | 55 | 10¹¹ |
| Right | — | — | — | 0.60 | 26 | 10¹¹ |
| parotid | | | | | | |
| Right | — | — | — | 0.60 | 26 | 10¹¹ |
| submandibular | | | | | | |

After preprocessing by the CPLEX solver, the model LP_{PWL} contained 538,334 constraints, 661,490 variables and 2,920,435 nonzero elements in the constraint matrix. The time needed to find the globally optimal solution was 302.5 s on a 2.8 GHz Pentium 4 laptop computer with 2 GB of RAM. The model LP_{PWL+CVaR} contained 562,694 constraints, 685,850 variables and 3,177,467 nonzero elements in the constraint matrix. The time needed to find the globally optimal solution was 425 s.

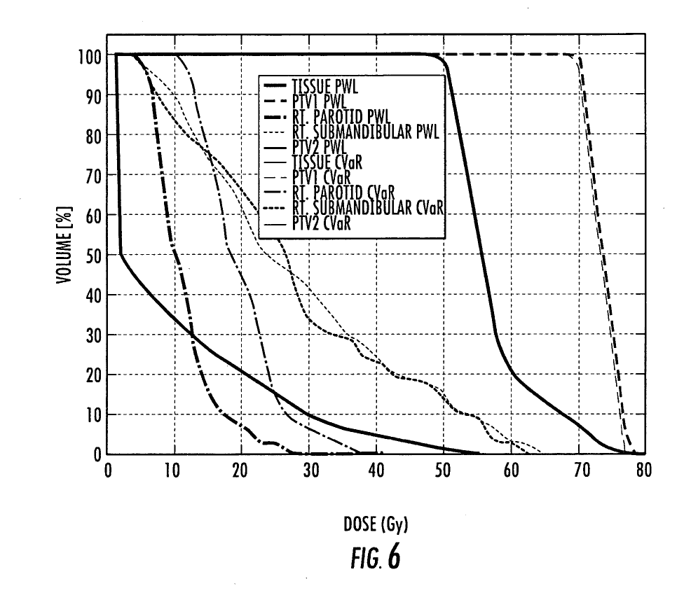

FIG. 6 shows cumulative DVHs for targets, spared salivary glands and tissue for both solutions. It was found that, in both cases, nearly all of our planning goals were satisfied. In the solution to the LPPWL model, target coverage was >95% for both target volumes at their prescription doses, with 100% coverage at 69 Gy for PTV1 and 99.6% coverage at 46.5 Gy for PTV2. The maximum dose to PTV1 was 78 Gy. Both contralateral salivary glands were spared, with 1% of the right parotid and 37.8% of the right submandibular gland receiving 30 Gy or higher. The spinal cord received a maximum dose of 47 Gy, with 1.2% exceeding 45 Gy. The maximum dose in the brainstem was 32 Gy. The unspecified tissue had 1.4% of its volume exceeding 50 Gy, and less than 0.1% of its volume exceeding 57 Gy. The mandible had 1% of its volume exceeding 70 Gy, and a maximum dose of 77 Gy. In the solution to the LPPWL+CVaR model, target coverage was slightly better, with 100% coverage at 69 Gy for PTV1, and 99.8% coverage at 46.5 Gy for PTV2. The maximum dose to PTV1 was 77 Gy. Both contralateral salivary glands were spared, with 3% of the right parotid and 35.8% of the right submandibular gland receiving 30 Gy or higher. The mean doses to these glands were 18 Gy and 28 Gy, respectively. The spinal cord received a maximum dose of 45 Gy. The maximum dose in the brainstem was 34 Gy. The unspecified tissue had 1.7% of its volume exceeding 50 Gy, and less than 0.1% of its volume exceeding 57 Gy. The mandible had 0.7% exceeding 70 Gy, and a maximum dose of 76 Gy.

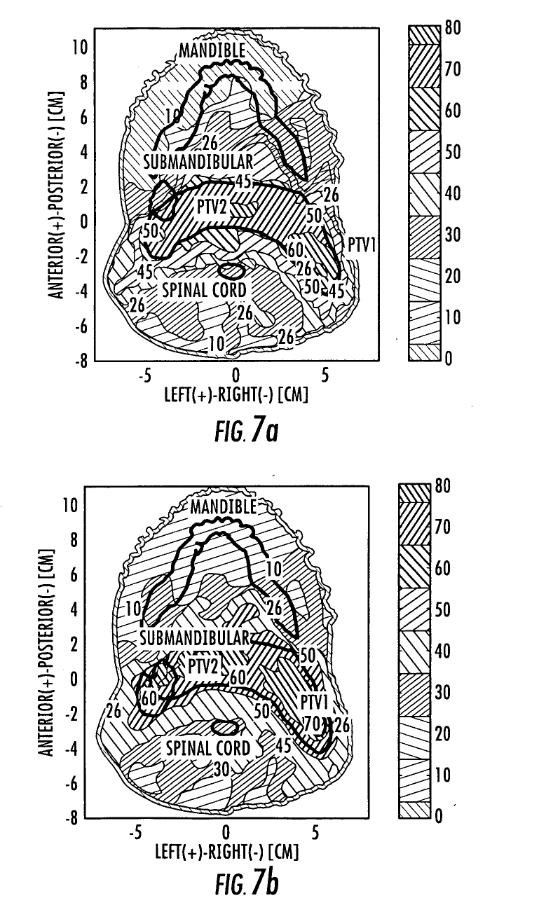

FIG. 7 illustrates the nearly equivalent salivary gland sparing obtained using the PWL voxel-based objective function or the CVaR dose-volume constraints described herein. Both contralateral salivary glands were spared with significantly less than the imposed limit of 50% at 30 Gy or higher. Noting that approximately 80% of the voxels in the model are in unspecified tissue of the case, the use of a coarser dose grid for this structure was investigated. Lowering the dose grid resolution from a 3 mm isotropic voxel grid to a 6 mm isotropic dose grid for only the unspecified tissue structure it was found that, after preprocessing by the CPLEX solver, the LP_{PWL} model with the reduced unspecified tissue resolution contained 30 201 constraints, 166,514 variables and 701,496 nonzero elements in the constraint matrix. The time needed to find the globally optimal solution was 52.9 s on a 2.8 GHz Pentium 4 computer with 1 GB of RAM. The LP_{PWL+CVaR} model with the reduced unspecified tissue resolution contained 190,874 constraints, 221,075 variables and 1,125,042 nonzero elements in the constraint matrix.

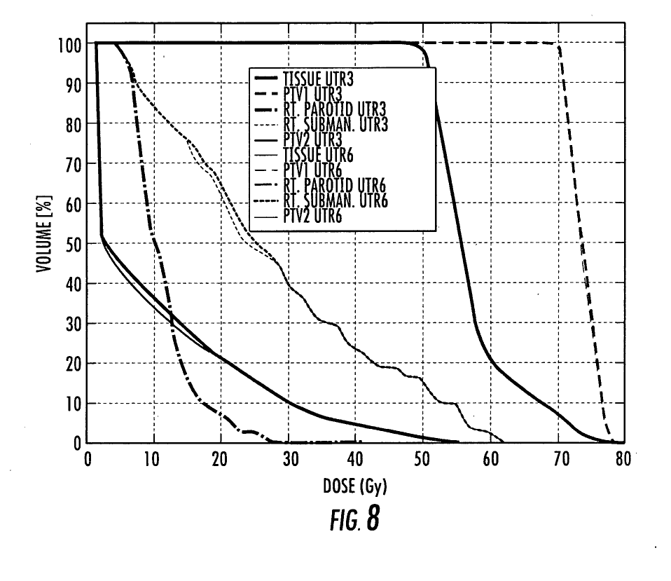

The time needed to find the globally optimal solution was 125.8 s. FIG. 8 demonstrates that the solutions of these models are nearly identical for the LPPWL model. Similar results were found for the LP_{PWL+CvaR} model.

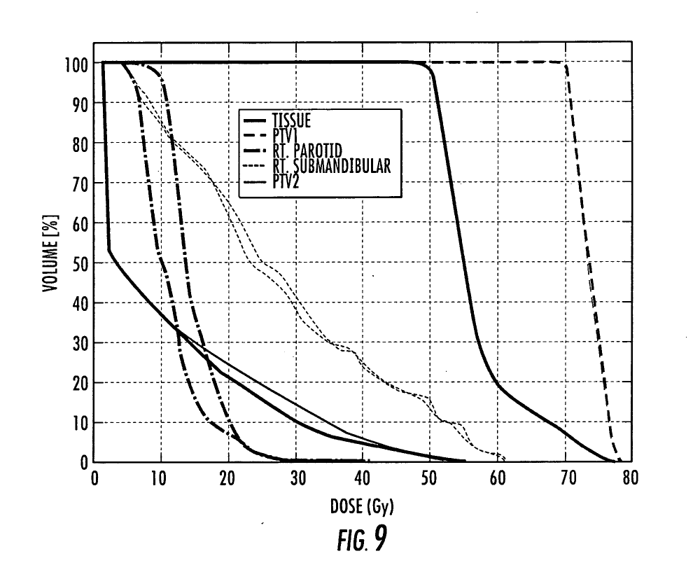

The influence of the number of segments used to approximate the nonlinear objective functions was then investigated in both models by doubling and quadrupling the number of segments while using the reduced unspecified tissue resolution. Increasing the number of segments does not increase the number of constraints in the model. Doubling increased the number of variables in the LPPWL model to 291,140, and the number of nonzero elements in the constraint matrix to 826,122, and the computation time to 65 s. Quadrupling increased the number of variables to 540,392, and the number of nonzero elements in the constraint matrix to 1,075,374, and the computation time to 96.3 s. The computation time increases approximately linearly with the number of segments in the model. FIG. 9 demonstrates that there is essentially no difference between the obtained solutions using the LPPWL model. A small difference was linearly with the number of segments in the model. FIG. 9 demonstrates that there is essentially no difference between the obtained solutions using the LP_{PWL} model. A small difference was observed in the DVHs for unspecified tissue at lower doses. This difference is insignificant and would not change any clinical decisions. Similar results were found for the LP_{PWL+CVaR} model. Finally, the robustness of the parameters that were obtained for the single case discussed above were investigated using manual adjustment by applying the model to seven additional head-and-ases neck IMRT cases where definitive therapy and salivary gland sparing was desired. The reduced unspecified tissue voxel grid was again used. The objective function parameters in Tables 1 and 2 were scaled by the ratios of the number of voxels in corresponding structures, to ensure that the relative importance of each structure remains the same in each case.

The coefficients for the left parotid and submandibular glands were chosen equal to the ones for the right parotid and submandibular glands. It was only attempted to spare a salivary gland if it was sufficiently far away from PTV1 (>1 cm) and more than 50% of its volume was outside PTV2. The sizes and run times of these models are found in Table 3 below.

TABLE 3
Model size and run times.

| | | | | | Number of | | |
|---|---|---|---|---|---|---|---|
| Case | Model | Run time (s) | Constraints | Variables | nonzero elements | Number of voxels | Number of beamlets |
| 1 | PWL | 52.9 | 30 201 | 166 514 | 701 496 | 40 984 | 1 182 |
| | CVaR | 125.8 | 190 874 | 221 075 | 1 125 042 | | |
| 2 | PWL | 127.0 | 63 127 | 358 125 | 1 427 730 | 82 540 | 1 745 |
| | CVaR | 267.9 | 369 129 | 432 345 | 1 880 735 | | |
| 3 | PWL | 151.3 | 66 205 | 368 824 | 1 474 210 | 90 967 | 1 804 |
| | CVaR | 434.8 | 426 072 | 492 277 | 2 440 754 | | |
| 4 | PWL | 36.8 | 29 871 | 165 898 | 696 882 | 41 523 | 1 017 |
| | CVaR | 88.7 | 173 856 | 203 727 | 923 095 | | |
| 5 | PWL | 40.0 | 26 156 | 156 448 | 655 604 | 32 942 | 1 015 |
| | CVaR | 84.9 | 30 007 | 161 580 | 694 098 | | |
| 6 | PWL | 55.5 | 38 831 | 231 925 | 936 477 | 52 800 | 1 178 |
| | CVaR | 132.2 | 240 007 | 278 840 | 1 241 489 | | |
| 7 | PWL | 54.3 | 35 121 | 212 958 | 876 606 | 45 688 | 1 090 |
| | CVaR | 87.8 | 39 325 | 218 133 | 920 971 | | |
| 8 | PWL | 37.2 | 26 173 | 155 868 | 644 395 | 36 566 | 921 |
| | CVaR | 84.5 | 161 012 | 187 185 | 846 979 | | |

Note that the number of constraints scales with the number of voxels, which explains the reduction in model size when the reduced voxel grid for unspecified tissue is used. The number of variables scales with both the number of voxels and the number of segments used to approximate the PWL penalty functions. The running times of LPPWL ranged between 37 and 151 s, and applying the CVaR constraints increased the running time by a factor of 2-3. Tables 4 and 5 below display dose-volume data to compare to our criteria for all plans obtained using the LP_{PLW} and LP_{PLW+CVaR} models, respectively. In addition, mean-dose data for the salivary glands is provided for reference.

TABLE 4
UFORT automated treatment-planning results
for eight head-and-neck cases using LP_{PWL}.

| | Dose-volume | | | | | | | | |
|---|---|---|---|---|---|---|---|---|---|
| Structure | result | 1 | 2 | 3 | 4 | 5 | 6 | 7 | 8 |
| All | Pass/Fail | Pass | Pass | Pass | Pass | Fail | Pass | Pass | Pass |
| PTV1 | % [symbol] D_{Rx} | 95 | 95 | 95 | 95 | 95 | 95 | 95 | 95 |
| PTV1 | % [symbol] .93 D_{Rx} | 100 | 100 | 100 | 100 | 100 | 100 | 100 | 100 |
| PTV1 | % [symbol] 1.1 D_{Rx} | 0.5 | 0 | 1.2 | 0 | 0.9 | 1.5 | 0.2 | 0 |
| PTV2 | % [symbol] D_{Rx} | 97.2 | 98.6 | 98.0 | 97.1 | 97.3 | 98.1 | 97.6 | 93.5 |
| PTV2 | % [symbol] .93 D_{Rx} | 99.6 | 99.9 | 99.6 | 99.4 | 99.6 | 99.5 | 99.4 | 98.9 |
| Left parotid | % [symbol] 30 Gy | — | 27.1 | 7.4 | 35.2 | 56.5 | 9.9 | 47.3 | 7.0 |
| | Mean dose | | 15.8 | 11.2 | 27.2 | 36.8 | 16.3 | 29.3 | 14.8 |
| Right parotid | % [symbol] 30 Gy | 1.0 | 19.4 | 16.0 | 40.4 | 63.2 | — | 21.4 | 71.1 |
| | Mean dose | 10.4 | 12.9 | 15.7 | 27.8 | 34.9 | | 16.9 | 39.9 |
| Left submandibular | % [symbol] 30 Gy | — | — | 27.8 | — | — | 42.4 | — | 44.8 |
| | Mean dose | | | 24.1 | | | 26.2 | | 28.7 |
| Right submandibular | % [symbol] 30 Gy | 37.8 | — | 70.8 | — | — | — | 76.9 | — |
| | Mean dose | 27.0 | | 36.8 | | | | 47.3 | |
| Spinal cord | % [symbol] 45 Gy | 1.1 | 0 | 1.3 | 0 | 0 | 0 | 0 | 0 |
| Brainstem | % [symbol] 54 Gy | 0 | — | 0 | 0 | 1.9 | 0 | 0 | 0 |
| Tissue | % [symbol] 50 Gy | 1.4 | 1.8 | 1.5 | 1.2 | 1.1 | 1.3 | 1.5 | 1.0 |

TABLE 5
UFORT automated treatment-planning results for
eight head-and-neck cases using LP_{PWL+CVaR}.

| | Dose-volume | | | | | | | | |
|---|---|---|---|---|---|---|---|---|---|
| Structure | result | 1 | 2 | 3 | 4 | 5 | 6 | 7 | 8 |
| All | Pass/Fail | Pass | Pass | Pass | Pass | Fail | Pass | Pass | Pass |
| PTV1 | % [symbol] D_{Rx} | 95 | 95 | 95 | 95 | 95 | 95 | 95 | 95 |
| PTV1 | % [symbol] .93 D_{Rx} | 100 | 100 | 100 | 100 | 100 | 100 | 100 | 100 |
| PTV1 | % [symbol] 1.1 D_{Rx} | 1.8 | 0 | 0 | 0 | 0 | 1.3 | 0 | 0.7 |
| PTV2 | % [symbol] D_{Rx} | 98.9 | 96.1 | 98.5 | 96.4 | 96.6 | 97.4 | 98 | 96.7 |
| PTV2 | % [symbol] .93 D_{Rx} | 99.8 | 99.9 | 99.8 | 98.9 | 99.1 | 99.3 | 99.5 | 99.2 |
| Left parotid | % [symbol] 30 Gy | — | 25.6 | 14.2 | 18.9 | 31.4 | 11.8 | 29.8 | 9.9 |
| | Mean dose | | 15.5 | 17.7 | 21.2 | 27.2 | 17.2 | 25.0 | 20.9 |
| Right parotid | % [symbol] 30 Gy | 3.0 | 20.8 | 17.4 | 24.1 | 37.5 | — | 18.6 | 72.4 |
| | Mean dole | 18.0 | 14.5 | 18.8 | 22.6 | 27.4 | | 23.0 | 39.8 |
| Left submandibular | % [symbol] 30 Gy | — | — | 29.3 | — | — | 60.0 | — | 45.6 |
| | Mean dose | | | 23.9 | | | 32.0 | | 30.6 |
| Right submandibular | % [symbol] 30 Gy | 35.8 | — | 93.8 | — | — | — | 95.0 | — |
| | Mean dose | 28.0 | | 46.8 | | | | 46.6 | |
| Spinal cord | % [symbol] 45 Gy | 0.2 | 0 | 0 | 0 | 0 | 0 | 0 | 0 |
| Brainstem | % [symbol] 54 Gy | 0 | — | 0 | 0 | 2.5 | 0 | 0 | 0 |
| Tissue | % [symbol] 50 Gy | 1.4 | 1.8 | 1.5 | 1.2 | 1.1 | 1.3 | 1.5 | 1.0 |

In all tables, case 1 refers to the case described in more detail above. It was found that the model parameters for LP_{PWL} determined for case 1 produced excellent plans for seven out of the eight cases. Plan 5 failed to spare any salivary glands and failed to adequately spare. A novel linear programming approach to fluence map optimization for IMRT 3539 the brainstem. Strictly speaking, plan 8 failed to adequately cover PTV2 at 50 Gy, although 99.4% of the prescription dose for PTV2 covered 95% of the target. In all cases other than case 5, all criteria were satisfied to within 2% of their values (values that exceed this 2% limit are italicized in tables 4 and 5). Note that the plans were run in an automated fashion without manual adjustment of the problem parameters. Table 5 shows that when the LP_{PLW+CVaR} model was applied, sparing of both parotid glands was achieved for case 5. Although the brainstem in that plan was still not adequately spared, inspection of the 3D planning data revealed that PTV 1 and PTV2 came as close as 3 and 1 mm, respectively, to the brainstem, creating a situation where a clinical tradeoff would have to be made by a human user. Adopting a strategy of first running the LP_{PLW} model, and then running the LP_{PLW+CVaR} model only if not all criteria are satisfied would result in an efficient and effective methodology for arriving at plans of good clinical quality.

It is to be understood that while the invention has been described in conjunction with the preferred specific embodiments thereof, that the foregoing description as well as the examples which follow are intended to illustrate and not limit the scope of the invention. Other aspects, advantages and modifications within the scope of the invention will be apparent to those skilled in the art to which the invention pertains.

1. A method of determining a treatment plan for intensity modulated radiation treatment (IMRT), comprising the steps of:
dividing a three-dimensional volume of a patient into a grid of dose voxels, wherein at least a portion of said dose voxels are designated to belong to at least one target or to at least one critical structure;
modeling an ionizing radiation dose as delivered by a plurality of beamlets each having a beamlet intensity;
providing a non-linear convex voxel-based penalty function model for optimizing a fluence map, said fluence map defining said beamlet intensities for each of said plurality of beamlets, and
solving said model based on defined clinical criteria for said target and said critical structure using an interior point algorithm with dense column handling to obtain a globally optimal fluence map.

2. The method of claim 1, wherein said penalty functions are selected from the group consisting of piece-wise linear functions, convex non-linear functions, and piece-wise non-linear convex functions.

3. The method of claim 1, wherein said dense column handling comprises Sherman Morrison Woodbury decomposition or Shur decomposition.

4. The method of claim 1, further comprising the step of constraining said model with a dose-volume constraint to produce a constrained model.

5. The method of claim 4, wherein said dose-volume constraint bounds a mean value of a tail of a differential dose-volume histogram (DVH) for a structure within said patient comprising a portion of said grid of dose voxels.

6. The method of claim 4, wherein said dose volume constraint comprises a conditional value at risk (CVaR) constraint.

7. The method of claim 6, wherein said CVaR constraint includes an upper and lower bound constraints on said dose received by each of said voxels comprising a given target region within said patient.

8. The method of claim 6, wherein said CVaR constraint includes upper and lower bound constraints on a mean dose received by a structure within said patient comprising a portion of said dose voxels.

9. A system for delivering intensity modulated radiation treatment (IMRT), comprising:
an inverse treatment planning system comprising:
computing structure for dividing a three-dimensional volume of a patient into a grid of dose voxels, wherein at least a portion of said dose voxels are designated to belong to at least one target or to at least one critical structure and modeling an ionizing radiation dose as delivered by a plurality of beamlets each having a beamlet intensity and for implementing a non-linear convex voxel-based penalty function model for optimizing a fluence map, said fluence map defining said beamlet intensities for each of said plurality of beamlets, and for solving said model based on defined clinical criteria for said target and said critical structure using an interior point algorithm with dense column handling to obtain a globally optimal fluence map;
a radiation source for generating at least one radiation beam, said radiation source including structure to generate said plurality of beamlets, and
a multi-leaf collimator disposed between said radiation source and said patient, said collimator communicably connected to said computing structure, said collimator having a plurality of leafs for modifying said plurality of beamlets to deliver said globally optimal fluence map to said patient.

10. The system of claim 9, wherein said penalty functions are selected from the group consisting of piece-wise linear functions, convex non-linear functions, and piece-wise non-linear convex functions.

11. The system of claim 9, wherein said dense column handling comprises Sherman Morrison Woodbury or Shur decomposition.

12. The system of claim 9, wherein said model is constrained with a dose-volume constraint to produce a constrained model.

13. The system of claim 12, wherein said dose-volume constraint bounds a mean value of a tail of a differential dose-volume histogram (DVH) for a structure within said patient comprising a portion of said grid of dose voxels.

14. The system of claim 12, wherein said dose volume constraint comprises a conditional value at risk (CVaR) constraint.
